(19) United States
(12) Patent Application Publication
(10) Pub. No.: US 2015/0223974 A1
(43) Pub. Date: Aug. 13, 2015

(54) PATIENT HEAT EXCHANGE SYSTEM WITH TRANSPARENT WALL FOR VIEWING CIRCULATING REFRIGERANT

(71) Applicant: Zoll Circulation, Inc., San Jose, CA (US)

(72) Inventors: JEREMY THOMAS DABROWIAK, Redwood City, CA (US); Eric Peterson, Pacifica, CA (US)

(73) Assignee: Zoll Circulation, Inc., San Jose, CA (US)

(21) Appl. No.: 14/276,202

(22) Filed: May 13, 2014

Related U.S. Application Data
(63) Continuation-in-part of application No. 14/175,545, filed on Feb. 7, 2014.

(51) Int. Cl. A61F 7/10; F28F 3/12; F25B 1/00

(52) U.S. Cl. CPC A61F 7/10; F25B 1/005; F28F 3/12; A61F 2007/0001

(57) ABSTRACT

A heat exchange system for exchanging heat with working fluid from an intravascular heat exchange catheter or an external heat exchange pad includes a working fluid that circulates between the catheter or pad and a fluid cassette, and a refrigerant system that flows against the outer sides of cold plates between which the cassette is disposed. An outer wall of the system is transparent so that a person can view the refrigerant as it circulates, ensuring proper operation.

This is a continuation in part of and claims priority to U.S. patent application Ser. No. 14/175,545, filed Feb. 7, 2014.

### I. FIELD OF THE INVENTION

The present application relates generally to patient heat exchange systems with a transparent wall for viewing circulating refrigerant.

### II. BACKGROUND OF THE INVENTION

Patient temperature control systems have been introduced to prevent fever in patients in the neuro ICU due to suffering from sub-arachnoid hemorrhage or other neurologic malady such as stroke. Also, such systems have been used to induce mild or moderate hypothermia to improve the outcomes of patients suffering from such maladies as stroke, cardiac arrest, myocardial infarction, traumatic brain injury, and high intracranial pressure. Examples of intravascular heat exchange catheters are disclosed in U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, all of which are incorporated herein by reference.

External patient temperature control systems may be used. Such systems are disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference.

### SUMMARY OF THE INVENTION

A heat exchange system for exchanging heat with working fluid from an intravascular heat exchange catheter or an external heat exchange pad includes a refrigerant circuit configured for circulating refrigerant between a compressor and first and second cold plates between which a working fluid cassette is disposable. At least a first one of the cold plates has a transparent outer wall through which a person can view refrigerant flowing in the refrigerant circuit.

In example embodiments a cassette slot is defined between the cold plates for receiving the fluid cassette. A distance between the cold plates (e.g., the width of the slot) can be less than forty mils (0.040″). Plural fasteners may hold the outer wall onto metal structure of the first cold plate. The fasteners may be arranged in four rows, one respective row along each of four edges of the outer wall. The cold plates can be nearly square and can abut each other along left and right side walls. In examples, respective vertically elongated cassette frame receptacles are located immediately inboard of the respective side walls with the slot extending between the side walls and terminating at the receptacles, and the frame receptacles are wider than the slot. At least one cold plate may be formed with a serpentine passageway through which the refrigerant flows.

In another aspect, system includes two heat transfer plates parallel to each other and defining a slot between them configured for receiving a working fluid cassette through which working fluid flows to and from an intravascular catheter in a working fluid circuit. A refrigerant circuit supplies refrigerant to at least one of the plates to exchange heat therewith. The refrigerant circuit includes a compressor and may be the only fluid circuit in thermal contact with the working fluid circuit other than a bloodstream of a patient in which the catheter can be positioned. According to present principles, at least one of the heat transfer plates is at least partially transparent to permit viewing the refrigerant as it flows in the refrigerant circuit.

In another aspect, a method includes circulating refrigerant between a compressor and a cold plate, and enabling a person to view the refrigerant as it circulates. The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic view of a non-limiting system in accordance with the present invention;

FIG. 2 is a perspective view of an example working fluid cassette holder portion of a heat exchange system;

FIG. 3 is another view of the transparent half of the cassette holder shown in FIG. 2 omitting the fasteners and holes for clarity;

FIG. 4 is a perspective view of an example working fluid cassette configured to engage the cassette holder shown in FIGS. 2 and 3; and

FIG. 5 is a close up perspective view of the cassette shown in FIG. 4, illustrating an inlet tube extending partially down into the stretched membrane chamber, it being understood that an opposed outlet tube may be similarly disposed on the opposite side of the cartridge and that both the inlet and outlet tubes may extend any length down their respective sides in the cassette.

### DETAILED DESCRIPTION OF THE PREFERRED EMBODIMENT

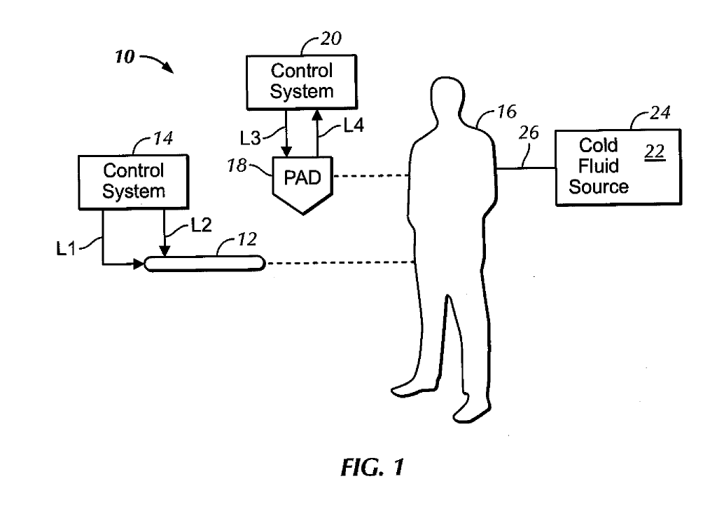

Referring initially to FIG. 1, in accordance with present principles, a system 10 may include an intravascular heat exchange catheter 12 controlled by a control system 14 to induce control patient temperature, e.g., to prevent the patient 16 from becoming febrile or to induce therapeutic hypothermia in the patient 16. In the catheter, working fluid (also referred to as “coolant”) such as but not limited to saline circulates (typically under the influence of a pump in the controller) in a closed loop from the control system 14, through a fluid supply line L1, through the catheter 12, and back to the system 14 through a fluid return line 12, such that no coolant enters the body. While certain preferred catheters are disclosed below, it is to be understood that other catheters can be used in accordance with present principles, including, without limitation, any of the catheters disclosed above or in the following U.S. patents, all incorporated herein by reference: U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by a pad control system 20 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia. The pad 18 may receive working fluid from the system 20 through a fluid supply line L3, and return working fluid to the system 20 through a fluid return line L4. Note that in some embodiments, the systems 14, 20 are established in a single assembly.

To cool the patient while awaiting engagement of the catheter 12 and/or pad 18 with the patient, cold fluid 22 in a cold fluid source 24 may be injected into the patient and in particular into the patient's venous system through a pathway 26. Without limitation, the pathway 26 may an IV line, the source 24 may be an IV bag, and the fluid 22 may be chilled saline, e.g., saline at the freezing point or slightly warmer. Or, the source may be a syringe, and the saline can be injected directly into the bloodstream of the patient.

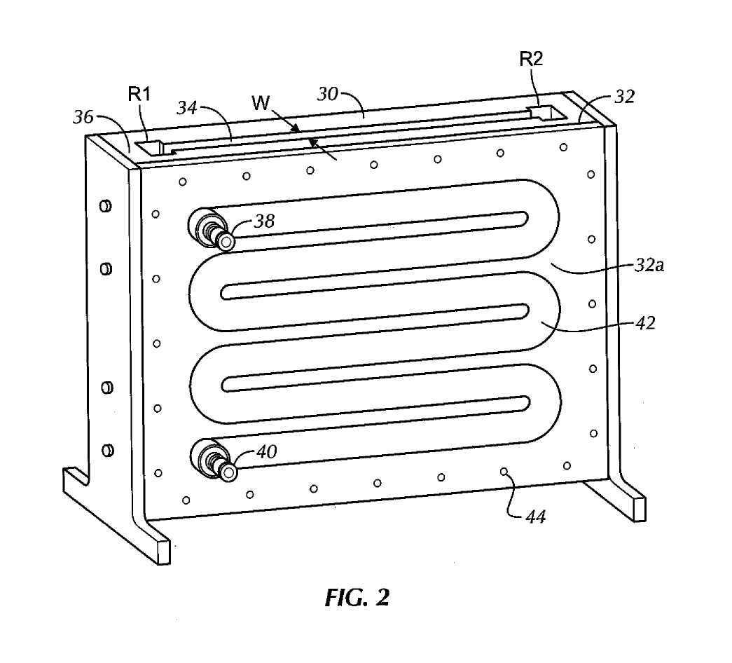

Now referring to FIG. 2, a portion of either of the heat exchangers in the control systems 14, 20 is shown which includes at least two cold plates 30, 32 defining a cassette slot 34 between them. In one embodiment, the width “W” of the slot 34 is less than forty mils (0.040″), and may be between twenty nine mils and thirty one mils (0.029″-0.031″) or may have a nominal slot width of 0.035″. In a specific example the width “W” may be thirty mils. In other embodiments, when a disposable heat exchange bag with serpentine channels is used, a larger gap between the cold plates may be used, e.g., 0.060″-0.120″ and more preferably 0.080″ to promote pumping saline through the bag without excessive backpressure.

The cold plates 30, 32 may be made of metal with the exception noted below, and can be rectilinear as shown and indeed may be nearly square. The cold plates 30, 32 may abut each other along left and right side walls 36, with elongated vertical cassette frame receptacles R1 and R2 being located immediately inboard of the respective side walls 36 and with the slot 34 extending between the walls 36 and terminating at the receptacles R1, R2 as shown. The frame receptacles R1, R2 are wider than the slot 36.

In the example shown, refrigerant inlet and outlet tubes 38, 40 extend through at least one of the cold plates 32 to communicate refrigerant from a compressor into a refrigerant passageway in the cold plate. Each cold plate may have its own refrigerant inlet and outlet tubes, or, in the embodiment shown, only one cold plate may be formed with refrigerant inlet and outlet tubes and the other cold plate either thermally coupled to the cold plate in which the refrigerant flows and/or receiving refrigerant from the other cold plate through passageways formed through one or both of the side walls 36.

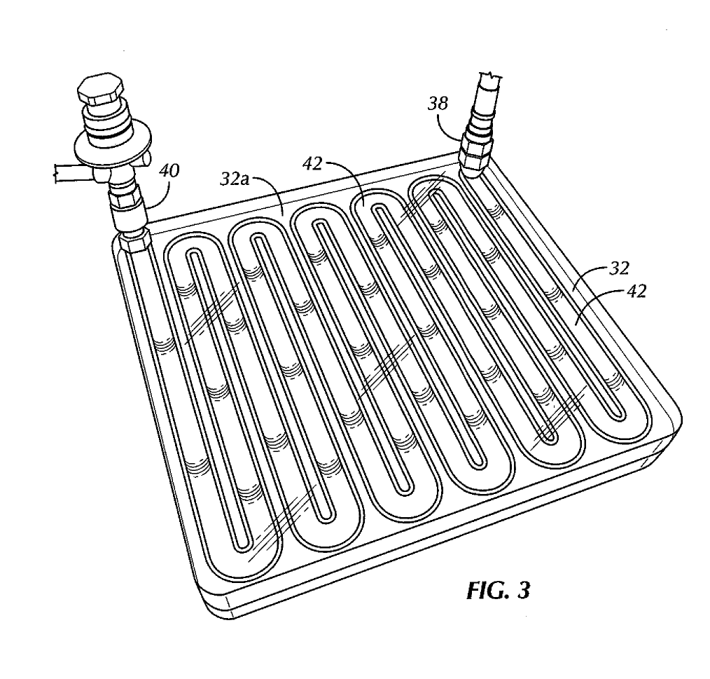

In the example shown in FIGS. 2 and 3, the outer wall of at least one of the cold plates is made at least partially (in the example shown, the entire wall is transparent) of transparent material such as hard plastic, e.g., plexiglass having a thickness of, e.g. one eighth of an inch, so that a person can view refrigerant flowing inside the cold plate to verify proper operation. For example, the outer wall 32a of the cold plate 32 may be transparent as shown.

Thus, as shown in FIGS. 2 and 3, owing to the transparent outer wall 32a of the cold plate 32, the refrigerant, in this non-limiting example constrained to flow in a serpentine refrigerant passageway 42, can be viewed during operation by a person looking at the outer wall 32a. The example refrigerant passageway that fluidly connects the refrigerant inlet 38 to the refrigerant outlet 40 may be serpentine-shaped as shown, or may be some other shape or pattern such as a herringbone pattern a wave pattern, etc. Alternatively, parallel channel passages may be used. For instance, ten one-inch wide channels may be formed in parallel, thereby achieving a 10×10″ surface area. Typically during operation the refrigerant bubbles as it transitions state while exchanging heat with the cassette and so the visual presentation to the viewer is of a flowing bubbling fluid.

Because the transparent outer wall 32a is exposed to fluid pressure from the inside, plural fasteners 44 (FIG. 2) may be used to secure the outer wall 32a to the remaining metal structure of the cold plate 32. Note that FIG. 3 does not show the fasteners for clarity, to better reveal the transparent view of the underlying refrigerant path.

The fasteners 44 shown in FIG. 2 may be threaded fasteners such as bolts that are received in corresponding threaded receptacles with which they are registered in the metal structure of the cold plate 32. For fortified securement, FIG. 2 shows that four rows of fasteners 44 may be used, a respective row of fasteners being arranged along each of the four edges of the wall 32a just inboard of the periphery of the wall. The spacing between adjacent fasteners may be, e.g., one half of an inch.

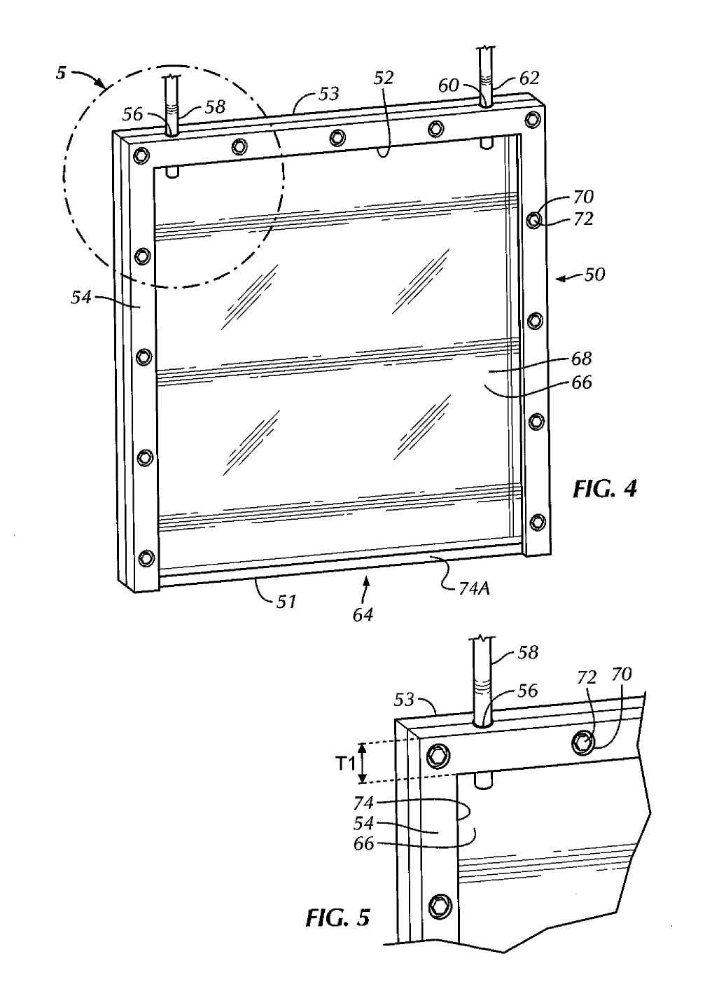

FIG. 4 shows an example working fluid cassette 50 according to present principles. The cassette 50 is configured to fit snugly into the slot 34 and cassette frame receptacles R1, R2 defined between the cold plates 30, 32. Working fluid such as saline from a patient-engageable heat exchange member such as the catheter 12 or pad 18 flows through the cassette 50 in operation, with the working fluid exchanging heat with the refrigerant in the cold plates. In example embodiments, the cassette 50 is a low cost single-use disposable item that can contain, e.g., sterile saline which circulates through the catheter 12. The cassette may be placed by a medical caregiver in the slot 34 between the cold plates 30, 32 and the membrane portion which defines a space or working fluid chamber through which the example saline flows inflates when the working fluid flows through it, achieving thermal contact with the cold plates 30, 32.

In the example shown, the cassette 50 includes a frame 52 defining a periphery and a preferably rectilinear opening bounded as shown on at least three sides by the periphery of the frame. In the non-limiting example shown, the frame includes an elongated parallelepiped-shaped top rail 53 and elongated parallelepiped-shaped left and right side rails 54 parallel to each other and perpendicular to the top rail 32. The example frame 52 has no bottom rail opposite the top rail. In any case, the example frame 52 is rectilinear and is configured for being closely received between the two cold plates 30, 32, with the side rails 54 slidably engageable with the frame receptacles R1, R2 between the cold plates 30, 32 and with the below-described membrane assembly passed through the slot 36 to be in close juxtaposition with the refrigerant channels in the cold plates.

In cross-references to FIGS. 4 and 5, the frame, in the example shown, the top rail 53 thereof, is formed with a fluid inlet 56 in which an inlet tube 58 has been disposed and a fluid outlet 60 in which an outlet tube 62 has been disposed. Both the inlet and outlet establish respective fluid passageways through the frame into the opening. The inlet and outlet tubes 58, 62 may be engaged with the fluid return and supply lines L3, L4 that are associated with the catheter 12. The tubes 58, 62 may terminate at just below the top rail 53 (FIG. 4), or they may extend any desired length down to the bottom of the assembly, i.e., the tubes 58, 62 may extend almost the entire length of the left and right side rails 54, ending just above the below-described bottom seam of the membrane assembly.

Indeed, a polymeric membrane assembly 64 is connected to the frame 52, blocking the opening that is bounded on three sides by the frame as shown. The membrane assembly includes a first membrane 66 that is parallel to and closely spaced from a second membrane 68, leaving a space there between which establishes a working fluid chamber. The fluid inlet 56 and fluid outlet 60 communicate with the space between the membranes 66, 68. At least one and preferably both of the membranes 66, 68 are disposed in tension in the opening. The space between the membranes is expandable when filled with working fluid.

In one example, each membrane is no more than two mils (0.002″) thick and more preferably is between one mil and two mils in thickness (0.001″-0.002″), inclusive. The example preferred membranes 66, 68 are co-extensive with the opening and like the opening are more or less square, with the length of top and bottom edges of the example membranes being approximately equal (within ±10% and more preferably within ±5%) of the lengths of the left and right edges of the membranes. Thus, the working fluid chamber between the membranes is also rectilinear and in the preferred embodiment no obstructions exist between the membranes, meaning the working fluid chamber is a complete rectilinear, more or less square chamber.

Owing to the thinness of the membranes 66, 68 and the closeness of the cold plates 30, 32 to each other and to the membrane assembly between them when the cassette is engaged with the cold plates, the system shown in the figures affords low impedance of heat transfer between the refrigerant circulating in the cold plates and the working fluid circulating between the membranes 66, 68. The working fluid chamber between the membranes inflates due to backpressure generated by working fluid flow, eliminating or reducing the need for a moving mechanism in the cold plates. Moreover, the narrow slot 34 between the two cold plates provides better heat transfer by reducing the conductive path length between the cold plates and the working fluid. The frame allows for ease of handling, such as insertion and removal of the cassette with/from the cold plates.

With respect to the example working fluid chamber between the membranes 66, 68 having a width-to-length aspect ratio near 1:1 (i.e., square or nearly so), the amount of backpressure required to induce working fluid flow through heat exchanger is reduced compared to a less square configuration. This reduces the amount of work that a working fluid pump must perform, which is desirable for two reasons. One, since the pump may be disposable, lower performance requirements translate into a lower cost disposable and quieter system. For instance, peristaltic roller pumps offer quiet operation and a low-cost disposable element, but operate most efficiently when only modest pressures are required. Two, lowering the working fluid pump work reduces the amount of heat transferred into the working fluid by the pump itself. Also, a low width/length aspect ratio results in slower working fluid velocity which reduces amount of mixing, but this otherwise desirable (from a heat exchange standpoint) effect is negligible in the present example system since the Reynolds numbers are typically <1000, suggesting a laminar flow regime. Furthermore, a low width/length aspect ratio significantly reduces the number of bends (or “corners”) in the fluid flow path. These bends are areas of mixing for the fluid which promotes heat transfer. Without them, a fluid boundary layer builds up. However, this effect is offset herein by maintaining a narrow slot between the cold plates. This way the primary heat transfer mechanism is by conduction, but the conduction path length (and therefore boundary layer) is small, resulting in a relatively high rate of heat transfer.

In preferred examples, the membranes 66, 68 are stretched under tension during assembly to the frame. This tension can be maintained over the shelf life of the product. Pretensioning minimizes wrinkles in material, which is beneficial because wrinkles can impede working fluid flow and create air gaps which reduce heat transfer between the working fluid and cold plates. Wrinkles can also complicate insertion of the membrane assembly into the narrow slot 34.

To establish pre-tensioning of the membranes, the frame may be made in halves and posts such as threaded fasteners 70 (FIG. 5) can extend transversely to one half of the frame, with the membranes 66, 68 being stretched over the posts and holes made in the membranes to receive the posts. The other half of the frame is then positioned to sandwich a rectilinear border portion 74 (only the innermost portion of which is shown in FIG. 5) of the membrane assembly between the frame halves, and a closure such as respective nuts 72 engaged with the posts 70 to hold the frame halves together with the membrane assembly held in tension between the frame halves. FIG. 4 shows that the working fluid chamber is closed off at the bottom by a bottom seam 74A of the membrane assembly, which is part of the border portion 74.

In the border portion 74, at least one and preferably more layers of polymer film may be used to reinforce the membranes 66, 68 to establish welded seams through which (at the sides of the membrane assembly) the post holes are formed, allowing for easier fabrication. By placing reinforcing layers on the border portion 74 only, the central “window” of the membrane assembly consists only of a single thin layer membrane ‘between the working fluid and one of the cold plates 30, 32 to minimize impeding heat transfer. A die-cut reinforcement layer may be used which reinforces the entire perimeter with one piece of material.

In some examples, the polymer membranes 66, 68 are highly stretchable, at least greater than 25% elongation. This allows the membranes to change from the empty flat state shown in FIGS. 4 and 5 to an inflated shape (within the slot 34 between the cold plates) without wrinkling. It also allows the membranes to easily conform to features on the faces of the cold plates.

Additionally, the membranes may be made of a material which can also be made into tubing. Tubes such as the inlet and outlet tubes 58, 62 shown in FIG. 4 can then be thermally welded (e.g., using RF sealing) to the membranes, which is more reliable and quicker than adhesive bonding. The membranes 66, 68 need not provide their own lateral support because the cold plates 32, 34 and frame provide the support for the inflated membrane assembly, allowing it to withstand the pressure generated as a result of working fluid flowing through between the membranes. Structural features may be located on the cold plates to optimize heat transfer. This can be economically advantageous because the cold plates are reusable components. Manifolds can be cut into the cold plates to even out the distribution of saline flow.

While the particular PATIENT HEAT EXCHANGE SYSTEM WITH TRANSPARENT WALL FOR VIEWING CIRCULATING REFRIGERANT is herein shown and described in detail, the scope of the present invention is to be limited by nothing other than the appended claims.

What is claimed is:

1. A heat exchange system for exchanging heat with working fluid from an intravascular heat exchange catheter or an external heat exchange pad, comprising:
a refrigerant circuit configured for circulating refrigerant between a compressor and first and second cold plates between which a working fluid cassette is disposable,
at least a first one of the cold plates having a transparent outer wall through which a person can view refrigerant flowing in the refrigerant circuit.

2. The system of claim 1, wherein the system is configured for circulating working fluid between the working fluid cassette and the catheter.

3. The system of claim 1, wherein the system is configured for circulating working fluid between the working fluid cassette and the pad.

4. The system of claim 1, wherein a cassette slot is defined between the cold plates for receiving the working fluid cassette.

5. The system of claim 1, wherein a distance between the cold plates is less than forty mils (0.040″).

6. The system of claim 1, comprising plural fasteners holding the outer wall onto metal structure of the first cold plate.

7. The system of claim 6, wherein the fasteners are arranged in four rows, one respective row along each of four edges of the outer wall.

8. The system of claim 1, wherein the cold plates abut each other along left and right side walls.

9. The system of claim 8, wherein respective vertically elongated cassette frame receptacles are located immediately inboard of the respective side walls with the slot extending between the side walls and terminating at the receptacles, the frame receptacles being wider than the slot.

10. The system of claim 1, wherein at least one cold plate is formed with a serpentine passageway through which the refrigerant flows.

11. System comprising:
two heat transfer plates parallel to each other and defining a slot between them configured for receiving a working fluid cassette through which working fluid flows to and from an intravascular catheter in a working fluid circuit;
a refrigerant circuit supplying refrigerant to at least one of the plates to exchange heat therewith, the refrigerant circuit including a compressor, the refrigerant circuit being the only fluid circuit in thermal contact with the working fluid circuit other than a bloodstream of a patient in which the catheter can be positioned, at least a first heat transfer plate being at least partially transparent to permit viewing the refrigerant as it flows in the refrigerant circuit.

12. The system of claim 11, wherein a distance between the heat transfer plates is less than forty mils (0.040″).

13. The system of claim 11, wherein the first heat transfer plate includes a transparent outer wall and the system comprises plural fasteners holding the outer wall onto metal structure of the first heat transfer plate.

14. The system of claim 13, wherein the fasteners are arranged in four rows, one respective row along each of four edges of the outer wall.

15. The system of claim 11, wherein the heat transfer plates abut each other along left and right side walls.

16. The system of claim 15, wherein respective vertically elongated cassette frame receptacles are located immediately inboard of the respective side walls with the slot extending between the side walls and terminating at the receptacles, the frame receptacles being wider than the slot.

17. The system of claim 11, wherein at least one heat transfer plate is formed with a serpentine passageway through which the refrigerant flows.

18. Method comprising:
circulating refrigerant between a compressor and a cold plate; and
enabling a person to view the refrigerant as it circulates.

19. The method of claim 18, comprising:
circulating working fluid between an intravascular heat exchange catheter and a fluid cassette disposed on contact with the cold plate to exchange heat between the refrigerant and the working fluid through the cold plate.

20. The method of claim 18, wherein the cold plate is a first cold plate and the method comprises circulating working fluid between the first cold plate and a second cold plate, at least one of the cold plates being at least partially transparent.
